(19) United States
(12) Patent Application Publication
(10) Pub. No.: US 2018/0066222 A1
(43) Pub. Date: Mar. 8, 2018

(54) DEVICE FOR MASSIVELY PARALLEL HIGH THROUGHPUT SINGLE CELL ELECTROPORATION AND USES THEREOF

(71) Applicant: The Regents of the University of California, Oakland, CA (US)

(72) Inventors: TUHIN SUBHRA SANTRA, Panskura (IN); MICHAEL A. TEITELL, Tarzana, CA (US); PEI-YU E. CHIOU, Los Angeles, CA (US)

(21) Appl. No.: 15/673,255

(22) Filed: Aug. 9, 2017

Related U.S. Application Data
(60) Provisional application No. 62/372,743, filed on Aug. 9, 2016.

(51) Int. Cl. C12M 1/42; C12M 1/12; C12Q 1/02; H01M 4/66

(52) U.S. Cl. CPC C12M 35/02; H01M 4/66; C12Q 1/025; C12M 23/04

(57) ABSTRACT

In various embodiments a Massively parallel Single-cell Electroporation Platform (MSEP) for low voltage, high efficiency delivery of extracellular materials into mammalian cells at an ultrahigh throughput of 10 million cells/min on a 1 cm² chip is provided. In certain embodiments MSEP is realized by a 3D silicon-based device with, e.g., 5,000 short vertical microfluidic channels in parallel. Single cells flowing through these channels are geometrically confined to regions with intense and localized electric fields where cells are electroporated. High efficiency delivery of calcium dyes, large-sized dextran proteins, and plasmids into mammalian cells to establish a range of sizes and compositions have been successfully accomplished with MSEP.

### CROSS-REFERENCE TO RELATED APPLICATIONS

This application claims benefit of and priority to U.S. Ser. No. 62/372,743, filed Aug. 9, 2016, which is incorporated herein by reference in its entirety for all purposes.

### STATEMENT OF GOVERNMENTAL SUPPORT

This invention was made with government support under Grant No. R01GM114188 awarded by the National Institutes of Health. The Government has certain rights in this invention.

### BACKGROUND

Electroporation is a well-established methodology for delivery of a variety of molecules into cells, including drugs, proteins and nucleic acids. The latter is highlighted by the electroporation-based delivery of DNA into cells to drive recombinant gene expression. The underlying principle is that an electric field generated by a high voltage pulse between two electrodes causes a transient dielectric breakdown of the plasma membrane of cells within the high intensity electric field, enabling the negatively-charged DNA to enter the cells. However, the process by which the DNA/cell membrane interface responds to the electric field to enable the DNA to enter the cell is not well understood.

The most common use for electroporation-based gene delivery is for molecular biology research, where simple plate electrodes within cuvettes enable routine transformation of competent cells on the bench. Electroporation-based gene delivery has subsequently been extended to in situ, ex vivo, and in vivo applications with development of specialized electroporation systems. These electroporation systems include a variety of electrode designs and voltage pulse shaping as part of optimized electroporation parameters, along with custom electroporation solutions and electrodes, with pulse intensity, pulse duration and repetition frequency being key parameters. These systems have proved effective in facilitating research in a range of tissues, including developmental neurobiology applications.

Conventional bulk electroporation is widely used but has been known to cause a high percentage of cell death and require high voltage sources. Microfluidic electroporation platforms can provide high delivery efficiency with high cell viability through better-controlled electric fields applied to cells. However, the throughput for microfluidic electroporation is typically orders of magnitude lower than conventional bulk approaches.

### SUMMARY

Various embodiments contemplated herein may include, but need not be limited to, one or more of the following:

Embodiment 1: A device for parallel single cell electroporation, said device comprising: a substrate comprising a plurality of through holes forming substantially parallel channels and a plurality of electrodes disposed so that each electrode comprising said plurality of electrodes intersects a subset of said plurality of holes and is configured to apply a voltage to or across the edges of said holes.

Embodiment 2: The device of embodiment 1, wherein said plurality of through holes comprises through holes disposed in a regular array and said plurality of electrodes comprises rows of electrodes disposed between rows of said holes each electrode intersecting a plurality of holes that comprises a row of holes.

Embodiment 3: The device according to any one of embodiments 1-2, wherein electrodes comprising said plurality of electrodes are covered with a dielectric material.

Embodiment 4: The device according to any one of embodiments 1-3, wherein said dielectric material is selected from the group consisting of an oxide, a photoresist, and polyimide.

Embodiment 5: The device according to any one of embodiments 1-4, wherein said dielectric material ranges in thickness from about 0.1 μm, or from about 1 μm up to about 10 μm, or up to about 8 μm, or up to about 6 μm, or up to about 5 μm, or up to about 4 μm, or up to about 3 μm, or up to about 2 μm.

Embodiment 6: The device according to any one of embodiments 1-5, wherein said plurality of holes form parallel channels having an average or median length ranging from about 1 μm up to about 100 μm, or from about 5 μm up to about 50 μm, or from about 10 μm up to about 40 μm.

Embodiment 7: The device according to any one of embodiments 1-6, wherein the average or median diameter of said plurality of holes ranges from about 5 μm up to about 50 μm, or from about 10 μm up to about 40 μm, or from about 15 μm up to about 30 μm, or up to about 20 μm.

Embodiment 8: The device according to any one of embodiments 1-7, wherein said through holes are configured to contain no more than 15 cells, or no more than 10 cells, or no more than 5 cells, or no more than 4 cells, or no more than 3 cells, or no more than 2 cells, or only one cell at a time.

Embodiment 9: The device according to any one of embodiments 1-8, wherein said device comprises at least 500 through holes, or at least 1000 through holes, or at least 2000 through holes, or at least 3000 through holes, or at least 4,000 through holes, or at least 5,000 through holes, or at least 6000 through holes, or at least 7,000 through holes, or at least 8,000 through holes, or at least 9,000 through holes, or at least 10,000 through holes, or at least 15,000 through holes, or at least 20,000 through holes, or at least 50,000 through holes, or at least 100,000 through holes, or at least 250,000 through holes, or at least 500,000 through holes, or at least 750,000 through holes, or at least 1,000,000 through holes.

Embodiment 10: The device according to any one of embodiments 1-9, wherein said through holes are disposed in an area ranging from about 0.5 cm², or from about 1 cm², up to about 10 cm², or up to about 8 cm², or up to about 6 cm², or up to about 5 cm², or up to about 4 cm², or up to about 3 cm², or up to about 2 cm², or up to about 1.5 cm².

Embodiment 11: The device according to any one of embodiments 1-10, wherein said device comprises at least about 500 holes/cm², or at least about 1000 holes/cm², or at least about 2000 holes/cm², or at least about 3000 holes/cm², or at least about 4,000 holes, or at least about 5,000 holes/cm², or at least about 6000 holes/cm², or at least about 7, or 000 holes/cm², or at least about 8,000 holes/cm², or at least about 9,000 holes/cm², or at least about 10,000 holes/cm², or at least about 15,000 holes/cm², or at least about 20,000 holes/cm², or at least about 25,000 holes/cm², or at least about 30,000 holes/cm², or at least about 35,000 holes/cm², or at least about 40,000 holes/cm².

Embodiment 12: The device according to any one of embodiments 1-11, wherein said substrate comprises a silicon substrate.

Embodiment 13: The device according to any one of embodiments 1-12, wherein said electrodes comprise a metal or metal alloy.

Embodiment 14: The device according to any one of embodiments 1-12, wherein said electrodes comprise a material selected from the group consisting of gold, silver, copper, graphite, titanium, brass, platinum, graphene, indium tin oxide (ITO), and carbon nanotube(s).

Embodiment 15: The device according to any one of embodiments 1-14, wherein the width of said electrode ranges from about 5 μm, or from about 10 μm, or from about 15 μm, or from about 20 μm up to about 500 μm, or from about 20 μm, or from about 30 μm, or from about 40 μm, or from about 50 μm up to about 500 μm, or up to about 400 μm, or up to about 300 μm, or up to about 200 μm, or up to about 150 μm.

Embodiment 16: The device according to any one of embodiments 1-15, wherein thickness of said electrode ranges from about 0.01 μm, or from about 0.05 μm, or from about 0.1 μm, or from about 0.2 μm, or from about 0.5 μm, or from about 1 μm, or from about 2 μm, or from about 3 μm, or from about 4 μm, or from about 5 μm, or from about 10 μm up to about 100 μm, or up to about 50 μm, or up to about 40 μm, or up to about 30 μm, or up to about 20 μm.

Embodiment 17: The device according to any one of embodiments 1-16, wherein said device further comprises a supporting structure comprising passages configured to permit fluid passage through said supporting structure and into said plurality of holes.

Embodiment 18: The device of embodiment 17, wherein said supporting structure comprises a honeycomb structure disposed on said substrate so that cells to be transfected pass through said honeycomb structure before entering holes comprising said plurality of through holes.

Embodiment 19: The device according to any one of embodiments 17-18, wherein the thickness of said supporting structure/honeycomb ranges from about 10 μm, or from about 20 μm, or from about 50 μm, or from about 100 μm up to about 500 μm, or up to about 400 μm, or up to about 300 μm, or up to about 200 μm, or up to about 150 μm.

Embodiment 20: The device according to any one of embodiments 17-19, wherein the average channel diameter of said supporting structure/honeycomb ranges from about 20 μ, or from about 30 μm, or from about 40 μm, or from about 40 μm, up to about 200 μm, or up to about 150 μm, or up to about 100 μm.

Embodiment 21: The device according to any one of embodiments 1-20, wherein:

said electrodes are disposed as a first layer on the substrate comprising said plurality of holes;

a dielectric layer is disposed on the top of said electrodes; and

said honeycomb is comprises a second layer disposed on the opposite side of said substrate that the side on which said electrodes are disposed.

Embodiment 22: The device according to any one of embodiments 1-21, wherein said electrodes are operably coupled to a power supply.

Embodiment 23: The device of embodiment 22, wherein said power supply provides a voltage ranging from about 1V, or from about 2V, or from about 3V, or from about 4V, or from about 5V up to about 50V, or up to about 40V, or up to about 30V, or up to about 20V, or up to about 15V.

Embodiment 24: The device supply according to any one of embodiments 22-23, wherein said power supply is configured to provide a DC voltage.

Embodiment 25: The device supply according to any one of embodiments 22-23, wherein said power supply is configured to provide an AC voltage.

Embodiment 26: The device of embodiment 25, wherein said power supply is configured to provide an AC voltage as a square wave.

Embodiment 27: The device of embodiment 25, wherein said power supply is configured to provide an AC voltage as a sine wave.

Embodiment 28: The device according to any one of embodiments 25-27, wherein said AC voltage ranges in frequency from about 10 Hz, or from about 100 Hz, or from about 1 kHz, or from about 10 kHz, up to about 1 MHz, or up to about 5 MHz, or up to about 10 MHz, or up to about 50 MHz.

Embodiment 29: The device according to any one of embodiments 1-28, wherein said device is in fluid communication with a chamber contain cells to be electroporated.

Embodiment 30: The device according to any one of embodiments 1-29, wherein said device is in fluid communication with a chamber containing a reagent (cargo) that is to be electroporated into said cells.

Embodiment 31: The device of embodiment 30, wherein said chamber containing cells to be electroporated and said chamber containing a cargo are different chambers that are in fluidic communication with each other.

Embodiment 32: The device of embodiment 30, wherein said chamber containing cells to be electroporated and said chamber containing a cargo are the same chamber.

Embodiment 33: The device according to any one of embodiments 30-32, wherein said chamber(s) are pressurized to force fluid containing said cells through said plurality of holes.

Embodiment 34: The device according to any one of embodiments 30-33, wherein said chamber(s) are chambers of a syringe or syringe pump.

Embodiment 35: A method of making an electroporation device according to any one of embodiments 1-21, said method comprising:

providing a substrate; backside etching of said substrate to form a honeycomb structure;

patterning and deposition of said plurality of electrodes on the front side surface of said substrate; and

etching through holes through said substrate and into the honeycomb structure.

Embodiment 36: The method of embodiment 35, wherein said substrate is a plastic substrate.

Embodiment 37: The method of embodiment 35, wherein said substrate is a silicon substrate.

Embodiment 38: The method according to any one of embodiments 35-37, wherein said backside etching comprises reactive ion etching.

Embodiment 39: The method of embodiment 38, wherein said reactive ion etching comprises FDRIE.

Embodiment 40: The method according to any one of embodiments 35-39, wherein said patterning and deposition comprises patterning a photoresist to define the electrodes, and vapor deposition to deposit the material comprising said electrodes.

Embodiment 41: The method according to any one of embodiments 35-40, wherein said etching through holes comprises deep reactive ion etching (DRIE).

Embodiment 42: The method according to any one of embodiments 35-41, wherein said method comprising depositing a dielectric layer on top of said electrodes.

Embodiment 43: A method of delivering a cargo into a plurality of cells, said method comprising:

providing cells in solution containing the cargo that is to be electroporated into said cells; and

passing said cells through the plurality of through holes in a device according to any one of embodiments 1-34, while applying a voltage to said electrodes whereby said cargo is electroporated into said cells.

Embodiment 44: The method of embodiment 43, wherein said passing cells through said plurality of holes comprises pressurizing said solution to drive said solution containing said cells through the plurality of holes.

Embodiment 45: The method of embodiment 44, wherein said pressure is applied using a syringe.

Embodiment 46: The method of embodiment 45, wherein said pressure applied using a syringe pump.

Embodiment 47: The method of embodiment 44, wherein said pressure is applied using a peristaltic pump.

Embodiment 48: The method of embodiment 44, wherein said pressure is applied using a hand pump.

Embodiment 49: The method of embodiment 44, wherein said pressure is applied using a gravity feed.

Embodiment 50: The method according to any one of embodiments 43-49, wherein said voltage ranges from about 1V, or from about 2V, or from about 3V, or from about 4V, or from about 5V up to about 50V, or up to about 40V, or up to about 30V, or up to about 20V, or up to about 15V.

Embodiment 51: The method according to any one of embodiments 43-50, wherein said voltage is an applied DC voltage.

Embodiment 52: The method according to any one of embodiments 43-50, wherein said voltage is an applied AC voltage.

Embodiment 53: The method of embodiment 52, wherein said voltage ranges in frequency from about 10 Hz, or from about 100 Hz, or from about 1 kHz, or from about 10 kHz, up to about 1 MHz, or up to about 5 MHz, or up to about 10 MHz, or up to about 50 mHz.

Embodiment 54. The method according to any one of embodiments 52-53, wherein said voltage is applied as a square wave.

Embodiment 55: The method according to any one of embodiments 52-53, wherein said voltage is applied as a sine wave.

Embodiment 56: The method according to any one of embodiments 43-55, wherein said cargo comprises one or moieties selected from the group consisting of a dye, a nucleic acid (e.g., RNA, DNA), a protein (including, but not limited to, antibodies, intrabodies, enzymes (e.g., kinases, proteases, helicases, phosphorylates, etc.), signaling molecules, and the like), a vector (e.g., a plasmid, a phagemid, bacteriophage vector, cosmid, etc.), a natural chromosome or chromosome fragment, a synthetic chromosome or chromosome fragment, a virus particle, a bacterium, an intracellular fungus, an intracellular protozoan, an organelle, various particles (e.g., nanoparticles, polymeric particles, drug-carrying particles, quantum dots, etc.), small organic molecules, probes, and labels.

Embodiment 57: The method of embodiment 56, wherein two or more different cargos are delivered into a single cell.

Embodiment 58: The method of embodiment 57, wherein the components of a CRISPR Cas9 gene editing system are delivered into a cell.

Embodiment 59: The method of embodiment 56, wherein said cargo comprises a vector (e.g., a plasmid, a phagemid, a cosmid).

Embodiment 60: The method of embodiment 56, wherein said cargo comprises a virus particle.

Embodiment 61: The method of embodiment 56, wherein said cargo comprises a bacterium.

Embodiment 62: The method of embodiment 56, wherein said cargo comprises an organelle.

Embodiment 63: The method of embodiment 62, wherein said cargo comprises a cell nucleus.

Embodiment 64: The method of embodiment 62, wherein said cargo comprises a mitochondrium.

Embodiment 65: The method of embodiment 56, wherein said cargo comprises a chromosome or chromosome fragment.

Embodiment 66: The method of embodiment 56, wherein said cargo comprises an artificial chromosome.

Embodiment 67: The method according to any one of embodiments 43-66, wherein said cells comprise a plant cell, a yeast cell, an algal cell, a fungal cell, an invertebrate animal cell (e.g., an insect cell), and a vertebrate animal cell.

Embodiment 68: The method of embodiment 67, wherein said cells comprise mammalian cells.

Embodiment 69: The device of embodiment 67, wherein said cells comprise human cells.

Embodiment 70: The device of embodiment 67, wherein said cells comprise non-human mammalian cells.

Embodiment 71: The device according to any one of embodiments 68-70, wherein said cells comprise lymphocytes, or stem cells.

Embodiment 72: The device of embodiment 71, wherein said cells comprise stem cells selected from the group consisting of adult stem cells, embryonic stem cells, cord blood stem cells and induced pluripotent stem cells.

Embodiment 73: The device according to any one of embodiments 68-70, wherein said cells comprise differentiated somatic cells.

Embodiment 74: The method according to any one of embodiments 43-67, wherein said cells comprise cells from a cell line.

Embodiment 75: The device of embodiment 74, wherein said cells comprise cells from a cell line listed in Table 1.

Embodiment 76: The device of embodiment 74, wherein said cells comprise cells from a cell line selected from the group consisting of HeLa, National Cancer Institute's 60 cancer cell lines (NCI60), ESTDAB database, DU145 (prostate cancer), Lncap (prostate cancer), MCF-7 (breast cancer), MDA-MB-438 (breast cancer), PC3 (prostate cancer), T47D (breast cancer), THP-1 (acute myeloid leukemia), U87 (glioblastoma), SHSY5Y Human neuroblastoma cells, cloned from a myeloma, and Saos-2 cells (bone cancer).

Embodiment 77: The method according to any one of embodiments 43-76, wherein sad device is operated at a flow rate that ranges from about 0.1 mL/min, or from about 0.5 mL/min, or from about 1.0 mL/min up to about 20 mL/min, or up to about 15 mL/min, or up to about 10 mL/min, or up to about 5 mL/min, or up to about 4 mL/min, or up to about 3 mL/min, or up to about 2 mL/min, or up to about 1.5 mL/min, or at about 1.12 mL/min.

Embodiment 78: The method according to any one of embodiments 43-77, wherein said cells are provided in said device at a density ranging from about 10⁵ cells/mL up to about 10⁹ cells/mL, or from about 10⁶ cells/mL up to about 10⁸ cells/mL, or about 10⁷ cells/mL.

Embodiment 79: The method according to any one of embodiments 43-78, wherein said device transfects cells at a delivery efficiency of at least about 10%, or at least about 20%, or at least 30%, or at least about 40%, or at least about 50%, or at least about 60%, at least about 70%, or at least about 80%, or at least about 90%.

Embodiment 80: The method according to any one of embodiments 43-79, wherein said device transfects cells with a cell viability of at least about 40%, or at least about 50%, or at least about 60%, at least about 70%, or at least about 80%, or at least about 90%.

Embodiment 81: The method according to any one of embodiments 43-80, wherein said method delivers cargos in up to 10 million cells/min on a 1 cm² chip.

### BRIEF DESCRIPTION OF THE DRAWINGS

FIG. 1 schematically illustrates one embodiment of a Massively parallel Single-cell Electroporation Platform (MSEP). As illustrated, the device consists of a silicon chip fabricated on a silicon-on-insulator (SOT) wafer with a 10 μm thick device layer and a 300 μm thick substrate layer. More than 5,000 through-device-layer holes with a diameter of 15 μm were patterned on a 1 cm² chip. The substrate layer was etched into a honeycomb structure to provide a fluid connection between these holes and the syringe reservoir storing cells and extracellular materials to be delivered into the cells. On top of these holes are self-aligned, comb-shaped electrodes providing highly localized electric fields to create transient cell membrane pores in single cells to allow extracellular materials to diffuse into the cell cytosols. This compact 3D silicon microfluidic chip is attached directly onto a handheld syringe pump.

FIG. 2A shows the detailed MSEP chip fabrication process. FIG. 2B shows the numerically simulated electric field distribution near a delivery hole. FIG. 2C shows SEM images of an array of holes and self-aligned electrodes on MSEP.

FIG. 3, panels a-d, illustrates the delivery of a calcein dye into HeLa cells. Panel a) Fluorescence images of cells after delivery of calcein dye (live HeLa cells). PI dye is used to check cell viability post-delivery. Panel b) Example data of delivery efficiency quantified and validated by standard flow cytometry analysis. Panels c) & d) Delivery efficiency and cell viability at 10 kHz and 10 MHz electrical signals.

FIG. 4, panels a-d, shows a comparison of cargo delivery results for cells of different sizes. Panel a) A fluorescence image of HeLa cells delivered with Dextran 3000. Panel b) Delivery efficiency and cell viability at different voltages. Panel (c) Dextran 3000 delivery into THP-1 cells at 10V, 10 MHz. Panel d) Delivery efficiency and cell viability of THP-1 cells.

FIG. 5, panels A-D, illustrates the delivery of very large molecules into HeLa cells. Panel a) shows the results of delivering very large molecules, dextran (MW: ˜70,000 daltons), into HeLa cells. Panels b) and c) show the efficiency and viability (PI dye) quantified using flow cytometry. Panel d) Efficiency and viability at different flow rates. Results of high cell viability but low delivery efficiency of large sized molecules matches theoretical predictions.

FIG. 6, panels a-b, illustrates plasmid delivery (GFP-Pmax) into THP-1 cells using 500 μs square wave pulse with cell flow rate 0.416 ml/min. Panel a) Fluorescence image 1 day post-delivery. Panel b) Delivery efficiency quantified by flow cytometry.

### DETAILED DESCRIPTION

The introduction of foreign cargo into living cells is an important method in cell biology research and the development of therapeutics. Electroporation is a powerful technique for delivering different extracellular molecules, such as certain drugs, DNA, RNA, dyes, tracers and oligonucleotides into different cell lines and primary cells, as well as whole tissues and organisms.

Conventional bulk electroporation is widely used but has been known to cause a high percentage of cell death and require high voltage sources. Microfluidic electroporation platforms can provide high delivery efficiency with high cell viability through better-controlled electric fields applied to cells. However, the throughput for microfluidic electroporation is typically orders of magnitude lower than conventional bulk approaches. Provided herein is a compact, easy to use, massively parallel, single-cell electroporation platform (MSEP) that not only overcomes the throughput limitation of microfluidic-based approaches but also requires only low voltage sources for high efficiency electroporation with high cell viability.

Disclosed herein is a massively parallel high throughput single cell electroporation platform (aka MSEP) that can be readily used to deliver different size and composition cargo into cells with high transfer efficiency and high retained cell viability post-delivery.

### Electroporation Devices

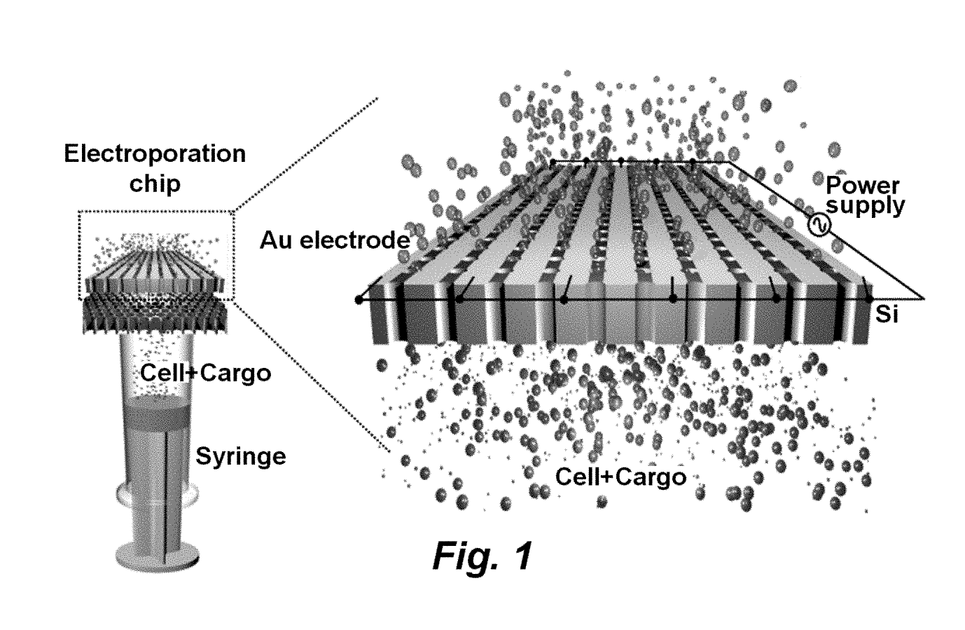

In one illustrative embodiment (see, e.g., FIG. 1, the device is 3 dimensional and silicon based with, e.g., 5,000 (or more) short vertical microfluidic channels (through holes) in parallel that can perform at an ultrahigh throughput to deliver various cargos in up to 10 million cells/min on a 1 cm2 chip. Compared with other microfluidic based electroporators, the device described herein provides several orders of magnitude higher throughput on a compact and easy to operate platform. Compared with conventional bulk electroporators, device described herein provides a low voltage, high efficiency, and high cell viability delivery method.

FIG. 1 schematically illustrates an embodiment of the MSEP device. As illustrated in this figure, the device is comprised of a chip (e.g., a silicon chip fabricated on a SOI wafer) with a 10 μm thick device layer and a 300 μm thick substrate layer. In the illustrated embodiment, more than 5,000 through-device-layer holes with a diameter of 15 μm are patterned on a 1 cm² chip. The substrate layer, when present, can be etched into a honeycomb structure to provide a fluid connection between these holes and a reservoir (chamber) such as a syringe reservoir storing cells and extracellular materials (cargos) to be delivered into the cells. On top of through holes are self-aligned, comb-shaped electrodes providing highly localized electric fields to create transient cell membrane pores in single cells to allow extracellular materials to diffuse into the cell cytosols.

It will be recognized that the configuration and dimensions shown are illustrative and need not be limiting. Using the teachings provided herein numerous other configurations will be available to one of skill in the art. Thus, in certain embodiments, a device for parallel single cell electroporation is provided where the device comprises substrate containing a plurality of through holes forming substantially parallel channels and a plurality of electrodes disposed so that each electrode comprising the plurality of electrodes intersects a subset of the plurality of holes and is configured to apply a voltage to or across the edges of the through-holes. In certain embodiments the plurality of through holes comprises through holes disposed in a regular array and the plurality of electrodes comprises rows of electrodes disposed between rows of the holes each electrode intersecting a plurality of holes that comprises a row of holes. In certain embodiments the electrodes comprising the plurality of electrodes are covered with a dielectric material (to reduce or prevent unnecessary power dissipation). In certain embodiments the dielectric material is selected from the group consisting of an oxide, a photoresist, and polyimide. In certain embodiments the dielectric material ranges in thickness from about 0.1 μm, or from about 1 μm up to about 10 μm, or up to about 8 μm, or up to about 6 μm, or up to about 5 μm, or up to about 4 μm, or up to about 3 μm, or up to about 2 μm.

In certain embodiments the plurality of holes form parallel channels having an average or median length ranging from about 1 μm up to about100 μm, or from about 5 μm up to about 50 μm, or from about 10 μm up to about 40 μm. In certain embodiments the average or median diameter of said plurality of holes ranges from about 5 μm up to about 50 μm, or from about 10 μm up to about 40 μm, or from about 15 μm up to about 30 μm, or up to about 20 μm. In certain embodiments the through holes are configured (sized) to contain no more than 15 cells, or no more than 10 cells, or no more than 5 cells, or no more than 4 cells, or no more than 3 cells, or no more than 2 cells, or only one cell at a time. In certain embodiments the device comprises at least 500 through holes, or at least 1000 through holes, or at least 2000 through holes, or at least 3000 through holes, or at least 4,000 through holes, or at least 5,000 through holes, or at least 6000 through holes, or at least 7,000 through holes, or at least 8,000 through holes, or at least 9,000 through holes, or at least 10,000 through holes, or at least 15,000 through holes, or at least 20,000 through holes, or at least 50,000 through holes, or at least 100,000 through holes, or at least 250,000 through holes, or at least 500,000 through holes, or at least 750,000 through holes, or at least 1,000,000 through holes. In certain embodiments the device comprises at least about 500 holes/cm², or at least about 1000 holes/cm², or at least about 2000 holes/cm², or at least about 3000 holes/cm², or at least about 4,000 holes, or at least about 5,000 holes/cm², or at least about 6000 holes/cm², or at least about 7, or 000 holes/cm², or at least about 8,000 holes/cm², or at least about 9,000 holes/cm², or at least about 10,000 holes/cm², or at least about 15,000 holes/cm², or at least about 20,000 holes/cm², or at least about 25,000 holes/cm², or at least about 30,000 holes/cm², or at least about 35,000 holes/cm², or at least about 40,000 holes/cm².

In certain embodiments the substrate comprises a silicon substrate or a polyimide substrate. In certain embodiments the electrodes comprise a metal or metal alloy. In certain embodiments the electrodes comprise a material selected from the group consisting of gold, silver, copper, graphite, titanium, brass, platinum, graphene, ITO, and carbon nanotube(s). In various embodiments the width of the electrodes ranges from about 5 μm, or from about 10 μm, or from about 15 μm, or from about 20 μm up to about 500 μm, or from about 20 μm, or from about 30 μm, or from about 40 μm, or from about 50 μm up to about 500 μm, or up to about 400 μm, or up to about 300 μm, or up to about 200 μm, or up to about 150 μm. In certain embodiments the thickness of the electrodes ranges from about 0.01 μm, or from about 0.05 μm, or from about 0.1 μm, or from about 0.2 μm, or from about 0.5 μm, or from about 1 μm, or from about 2 μm, or from about 3 μm, or from about 4 μm, or from about 5 μm, or from about 10 μm up to about 100 μm, or up to about 50 μm, or up to about 40 μm, or up to about 30 μm, or up to about 20 μm.

In certain embodiments, the device comprises supporting structure (e.g., a honeycomb structure as described above) that facilitates placement of the device in fluid communication with one or more chambers containing the cells to be electroporated and the cargo to be delivered into the cells. In certain embodiments the honeycomb structure is disposed on the substrate so that cells to be transfected pass through the honeycomb structure before entering holes comprising said plurality of through holes. In certain embodiments the thickness of said honeycomb ranges from about 10 μm, or from about 20 μm, or from about 50 μm, or from about 100 μm up to about 500 μm, or up to about 400 μm, or up to about 300 μm, or up to about 200 μm, or up to about 150 μm. In certain embodiments the average channel diameter of said honeycomb ranges from about 20μ, or from about 30 μm, or from about 40 μm, or from about 40 μm, up to about 200 μm, or up to about 150 μm, or up to about 100 μm. Typically, when present, the honeycomb structure is present to provide mechanical support. It will be recognized that other materials can be substituted to perform a similar function. For example, other materials such as photoresist, or other plastic or glass substrates can perform the same function.

It will be recognized that these dimensions, materials, and configurations are illustrative and not necessarily limiting. For example, the substrate material is not limited to silicon. Other materials such as polyimide and the like can be used as well. Using the teaching provided herein, numerous other device configurations will be available to one of skill in the art.

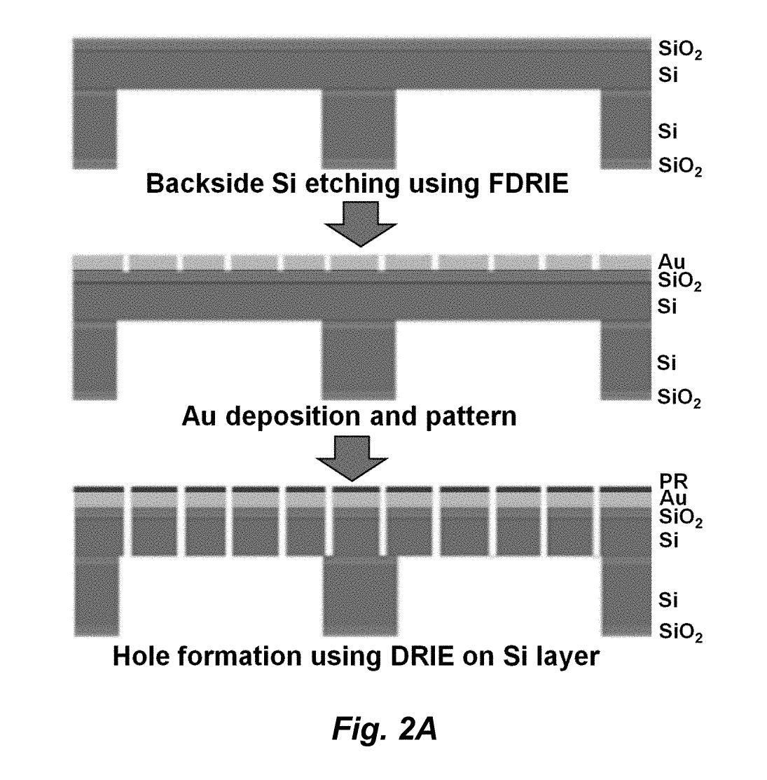

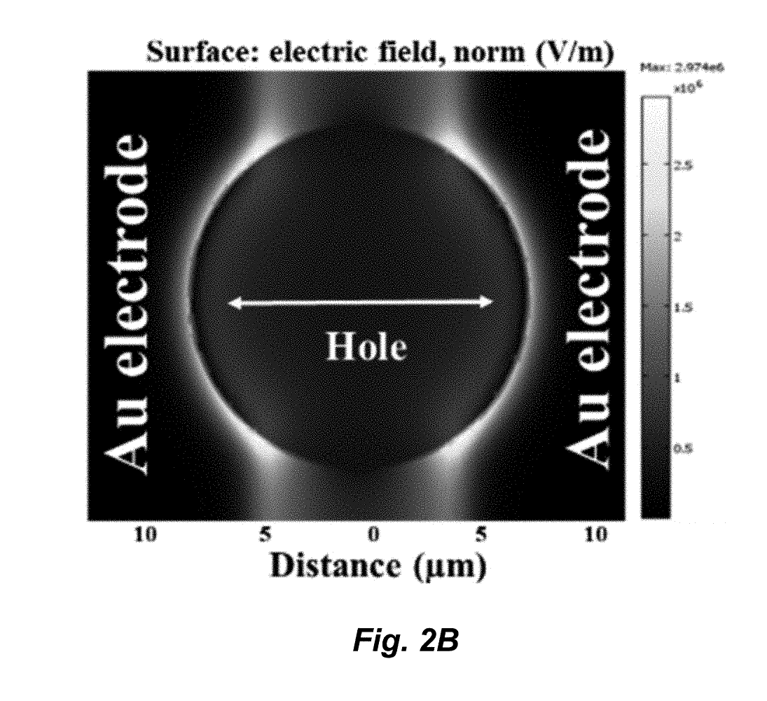

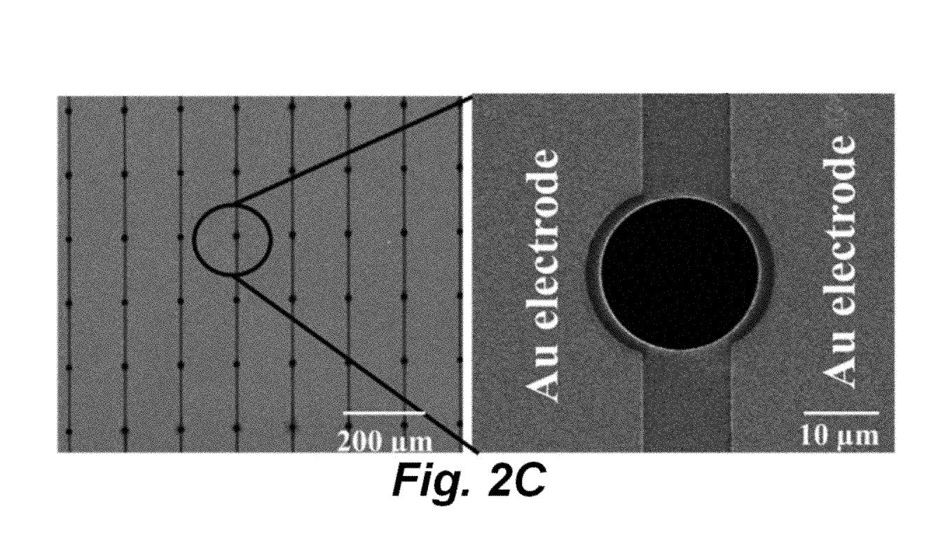

FIG. 2A illustrates one embodiment of a process for fabricating the MSEP device. In certain embodiments methods of making the MSEP device comprise providing a silicon substrate (e.g., an SOI substrate), backside etching of the substrate to form a honeycomb structure, patterning and deposition of a plurality of electrodes on the front side surface of the substrate; and etching through holes through the substrate and into the honeycomb structure. In certain embodiments the backside etching comprises fast deep reactive ion etching (FDRIE). In certain embodiments the patterning and deposition comprises patterning a photoresist to define the electrodes, and vapor deposition to deposit the material comprising said electrodes. In certain embodiments etching through holes comprises deep reactive ion etching (DRIE) of through holes.

FIG. 2B shows a numerically simulated electric field distribution near a delivery hole (substrate through hole). FIG. 2C shows SEM images of an array of holes and self-aligned electrodes on MSEP.

In certain embodiments the electroporation devices contemplated herein comprise the compact 3D silicon microfluidic chip attached directly onto a syringe or a syringe pump (e.g., a handheld syringe pump). The syringe pump, a hand pump, or other methods, can be used to pressurize the chamber (chamber containing cells and cargo) to drive the cells through the electroporation device.

It will be recognized that the device described above is illustrative and not limiting. Using teachings provided herein, devices comprising other configurations and materials will be available to one of skill in the art. By way of illustration, a silicon substrate is simply illustrative. Other materials that can be made to have similar membrane structures with through layer holes and metal electrode patterns can provide the same electroporation function. For example, in certain embodiments, one may simply drill an array of holes on a plastic sheet and deposit metal electrodes near these holes. This will function as well although it may not be as optimized as the particular embodiments illustrated herein.

### Methods of Delivering a Cargo Into a Cell

In certain embodiments methods of utilizing the electroporation device described herein to deliver a cargo into a plurality of cells are provided. In certain embodiments the method involves: 1) providing cells in a solution containing the cargo that is to be electroporated into said cells; and passing the cells through the plurality of through holes in the electroporation device described herein, while applying a voltage to the electrodes whereby the cargo is electroporated into said cells. In certain embodiments passing cells through said plurality of holes involves pressurizing the solution to drive the solution containing cells through the plurality of holes. In certain embodiments the pressure is applied using a syringe or syringe pump, or a peristaltic pump, or a gravity feed. In certain embodiments the applied voltage ranges from about 1V, or from about 2V, or from about 3V, or from about 4V, or from about 5V up to about 50V, or up to about 40V, or up to about 30V, or up to about 20V, or up to about 15V. In certain embodiments the voltage is an applied DC voltage. In certain embodiments the applied voltage is an AC voltage. In certain embodiments the AC voltage ranges in frequency from about 10 Hz, or from about 100 Hz, or from about 1 kHz, or from about 10 kHz, up to about 1 MHz, or up to about 5 MHz, or up to about 10 MHz, or up to about 50 MHz. In certain embodiments the AC voltage is applied as a square wave. In certain embodiments the AC voltage is applied as a sine wave.

In certain embodiments the cargo comprises a cargo as described below (e.g., one or moieties selected from the group consisting of a dye, a nucleic acid (e.g., RNA, DNA), a protein (including, but not limited to, antibodies, intrabodies, enzymes (e.g., kinases, proteases, helicases, phosphorylates, etc.), signaling molecules, and the like), a vector, a natural chromosome or chromosome fragment, a synthetic chromosome or chromosome fragment, a virus particle, a bacterium, an intracellular fungus, an intracellular protozoan, an organelle, various particles (e.g., nanoparticles, polymeric particles, drug-carrying particles, quantum dots, etc.), and the like. It will also be recognized that in certain embodiments two different cargos can be delivered into a cell using the devices and methods described herein. For example in certain embodiments, both a protein and a nucleic acid can be delivered into the same cell. Thus, for example, the methods can be used to deliver the components of a CRISPR Cas9 gene editing system (e.g., Cas9 enzyme, along with the crRNA and trRNA or along with a single guide RNA).

In certain embodiments the cell(s) to be transfected comprise a plant cell, a yeast cell, an algal cell, a fungal cell, an invertebrate animal cell (e.g., an insect cell), or a vertebrate animal cell.

In various embodiments the device is operated at a flow rate that ranges from about 0.1 mL/min, or from about 0.5 mL/min, or from about 1.0 mL/min up to about 20 mL/min, or up to about 15 mL/min, or up to about 10 mL/min, or up to about 5 mL/min, or up to about 4 mL/min, or up to about 3 mL/min, or up to about 2 mL/min, or up to about 1.5 mL/min, or at about 1.12 mL/min. In certain embodiments the cells are provided in said device at a density ranging from about 10⁵ cells/mL up to about 10⁹ cells/mL, or from about 10⁶ cells/mL up to about 10⁸ cells/mL, or about 10⁷ cells/mL. In certain embodiments the device transfects cells at a delivery efficiency of at least about 10%, or at least about 20%, or at least 30%, or at least about 40%, or at least about 50%, or at least about 60%, at least about 70%, or at least about 80%, or at least about 90%. In certain embodiments the device transfects cells with a cell viability of at least about 40%, or at least about 50%, or at least about 60%, at least about 70%, or at least about 80%, or at least about 90%. In certain embodiments the method delivers cargos in up to 10 million cells/min on a 1 cm² chip.

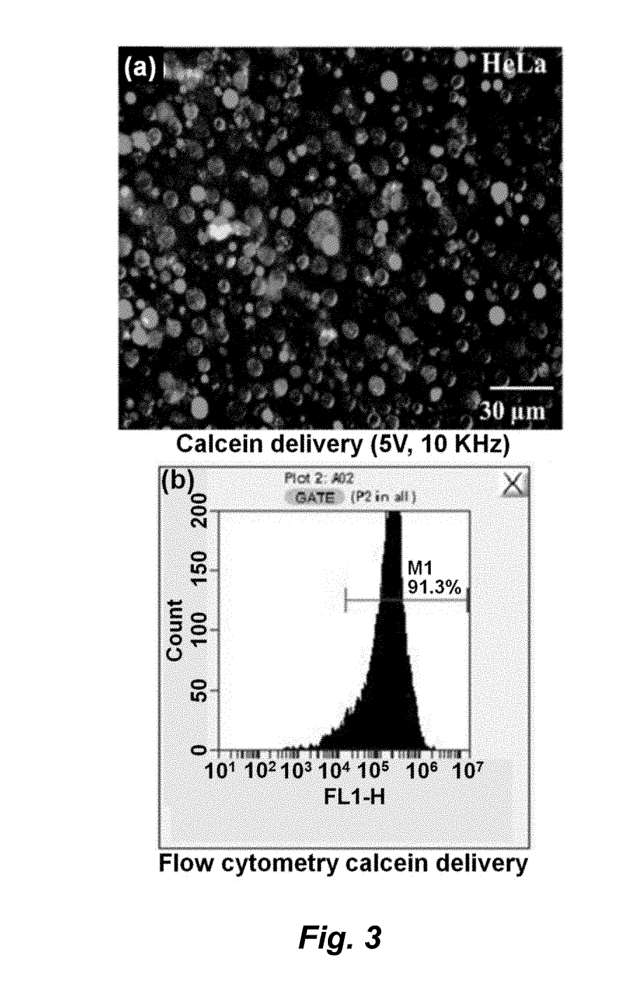

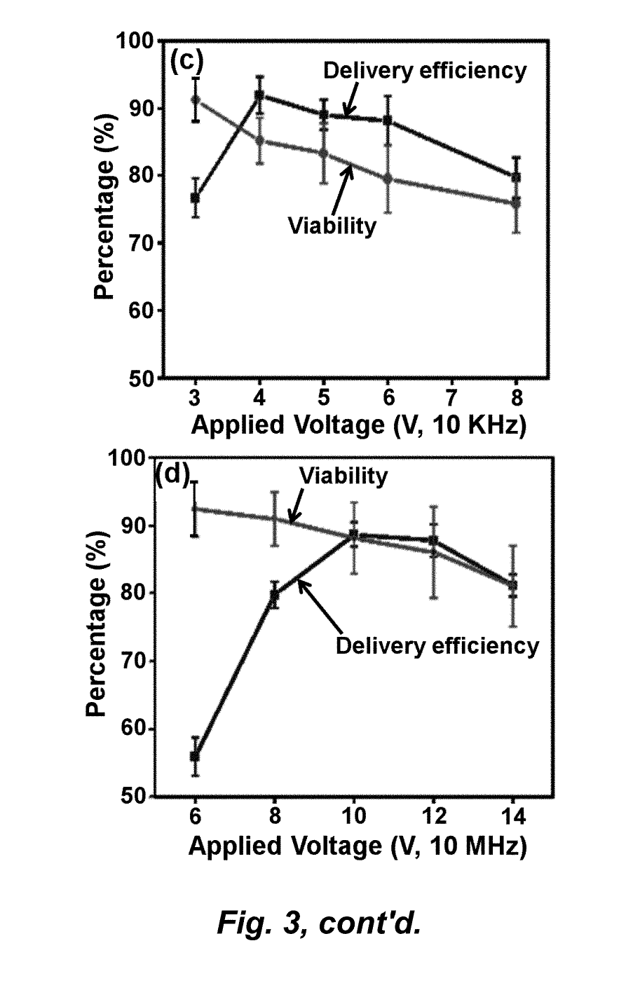

FIG. 3, panel a, shows the results of delivering a calcein dye into HeLa cells at a flow rate of 1.12 ml/min at a cell density of 10⁷ cells/ml. The delivery efficiency is quantified and validated by a commercial flow cytometer (FIG. 3, panel b). FIG. 3, panels c) and d) compare the results when applying electric signals at the kHz and MHz ranges. At an optimal condition (e.g., 10V, 10 MHz), 90% delivery efficiency and 90% cell viability has been achieved.

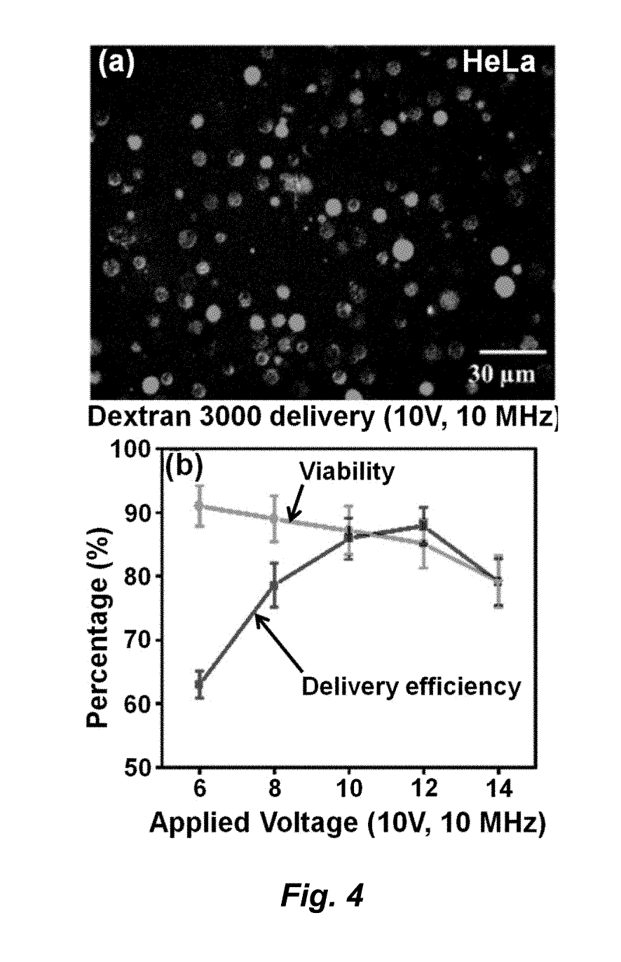

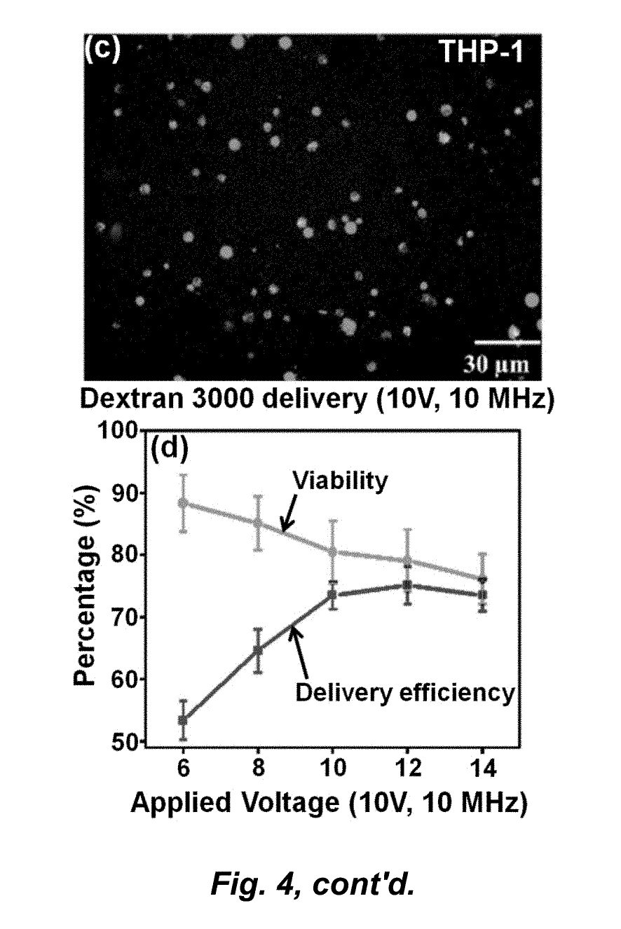

FIG. 4, panels a-d, compares the results of delivering dextran (MW: 3,000 daltons) into HeLa cells and THP-1 cells, whose size is smaller than HeLa. Close to 90% delivery efficiency and 90% viability was achieved in HeLa cells. However, the delivery efficiency to THP-1 cells decreased to about 73% due to the smaller cell size and higher possibility of passing through regions in a delivery hole with a lower electric field strength.

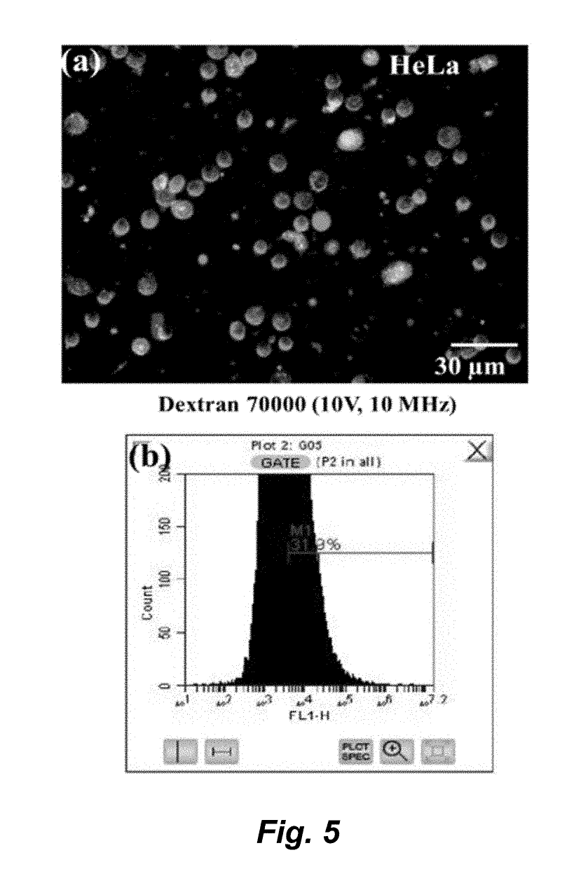

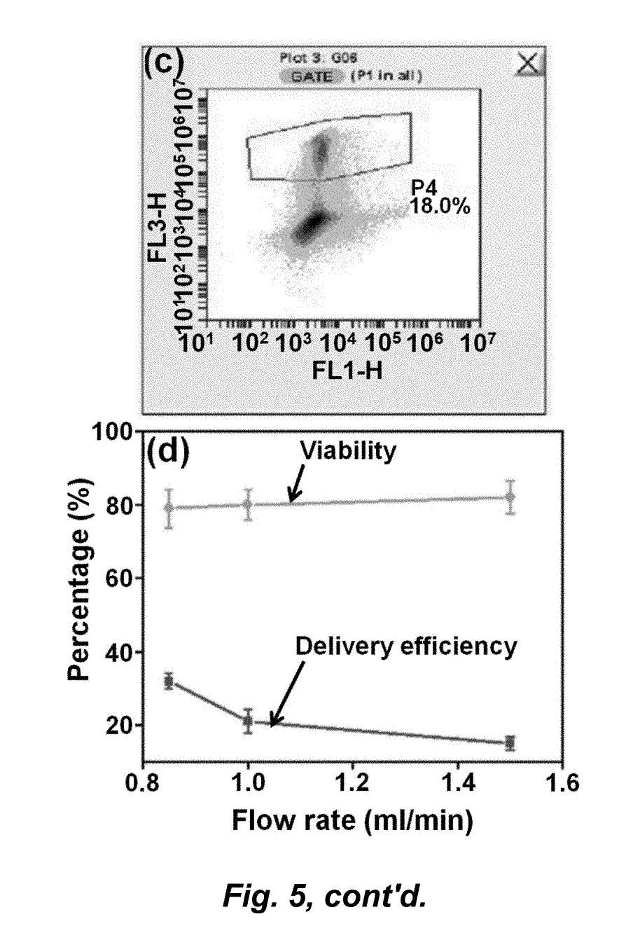

FIG. 5, panels a-d, shows the results of delivering very large molecules, dextran (MW: 70,000 daltons), into HeLa cells. The low delivery efficiency (<30%) matches the expectation that larger sized molecules diffuse more slowly into a cell's cytosol through small transient membrane pores generated by electroporation.

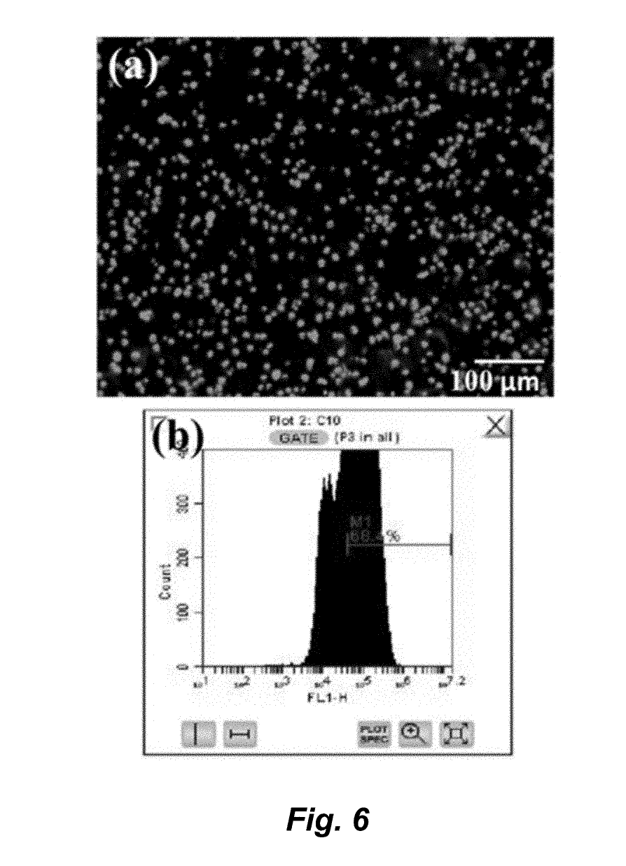

FIG. 6, panels a-b, shows a plasmid (GFP-Pmax) delivered into THP-1 cells with an applied 500 μs square wave pulse that resulted in 68% transfection efficiency and 79% cell viability one day following electroporation.

### Deliverable Materials (Cargo)

It is believed possible to deliver essentially any desired material into a cell using the electroporation devices and methods described herein. Such materials include, but are not limited to a nucleic acid (e.g., RNA, DNA), a protein (including, but not limited to, antibodies, intrabodies, enzymes (e.g., kinases, proteases, helicases, phosphorylates, etc.), signaling molecules, and the like), a vector (e.g., a plasmid, a phagemid, bacteriophage vectors, cosmids, etc.), a natural chromosome or chromosome fragment, a synthetic chromosome or chromosome fragment, a virus particle, a bacterium, an intracellular fungus, an intracellular protozoan, an organelle, various particles (e.g., nanoparticles, polymeric particles, drug-carrying particles, quantum dots, etc.), small organic molecules, probes, labels, and the like. It will also be recognized that in certain embodiments two different cargos can be delivered into a cell using the devices and methods described herein. For example in certain embodiments, both a protein and a nucleic acid can be delivered into the same cell. Thus, for example, the methods can be used to deliver the components of a CRISPR Cas9 gene editing system (e.g., Cas9 enzyme, along with the crRNA and trRNA, or along with a single guide RNA). In embodiments, the cargo comprises one or more moieties selected from the group consisting of a dye, a nucleic acid, an antibody, a vector, a natural chromosome or chromosome fragment, a synthetic chromosome or chromosome fragment, a virus particle, a bacterium, an intracellular fungus (e.g., Pneumocystis jirovecii, Histoplasma capsulatum, Cryptococcus neoformans, etc.), an intracellular protozoan (e.g., Apicomplexans (e.g., Plasmodium spp., Toxoplasma gondii, Cryptosporidium parvum), Trypanosomatids (e.g., Leishmania spp., Trypanosoma cruzi, etc.), and the like), and an organelle (e.g., a nucleus, a nucleolus, a mitochondrion, a chloroplast, a ribosome, a lysosome, and the like), an intracellular protozoan, an organelle (e.g., a nucleus, a nucleolus, a mitochondrion, a chloroplast, a ribosome, a lysosome, and the like).

In certain embodiments the cargo comprises a nucleus, and/or a chloroplast, and/or a nucleolus, and/or a mitochondrion.

In certain embodiments the cargo comprises a whole chromosome, or a chromosome fragment, or a synthetic chromosome (e.g., a BACs (bacterial artificial chromosome)). It is believed the devices and methods described herein can be used to deliver whole or partial natural or synthetic chromosomes. Similar to BACs, large chromosomes or chromosomal fragments that cannot be transduced into most cell types by previous methods can be transferred into cells by the method described herein, for example, inter alia, to establish models of human trisomy disorders (e.g., Down and Klinefelter syndromes).

In certain embodiments the cargo comprises intracellular pathogens, including but not limited to various bacteria, fungi, and protozoans. The transfection of various inanimate particles is also contemplated. Such particle include, but are not limited to quantum dots, surface-enhanced, Raman scattering (SERS) particles, microbeads, and the like.

It will be recognized that these cargos are intended to be illustrative and non-limiting. Using the teachings provided herein, numerous other cargos, especially large cargos, can be transfected into cells.

### Cell Types for Electroporation Using the Devices and Methods Described Herein

It is believed the electroporation devices and methods described herein can be used with essentially any cell having a cell membrane. In addition, in certain embodiments the methods and devices can also be used on cells having a cell wall. Accordingly, in various embodiments, it is contemplated that essentially any cell capable of electroporation, can be transfected using the electroporation devices and methods described herein. Thus, for example, suitable cells that can be transfected using the methods described herein include, but are not limited to plant cells, yeast cells, algal cells, fungal cells, an invertebrate animal cells (e.g., an insect cell), and vertebrate animals (including mammals and non-mammalian vertebrate cells). In certain embodiments the cells are mammalian cells (e.g., human cells, non-human mammalian cells), insect cells, fungal cells, or invertebrate cells.

Commonly, the methods described herein will be performed with mammalian cells including both human mammalian cells and non-human mammalian cells (e.g., non-human primates, canines, equines, felines, porcines, bovine, ungulates, largomorphs, and the like).

In certain embodiments, the cells that are to be electroporated include stem cells or committed progenitor cells. In certain embodiments the stem cells include adult stem cells, fetal stem cells, cord blood stem cells, acid-reverted stem cells, and induced pluripotent stem cells (IPSCs).

In certain embodiments the cells comprise lymphocytes or other differentiated somatic cells.

In certain embodiments the cells to be electroporated comprise cells from a cell line. Suitable cell lines include for example, HeLa, National Cancer Institute's 60 cancer cell lines (NCI60), ESTDAB database, DU145 (prostate cancer), Lncap (prostate cancer), MCF-7 (breast cancer), MDA-MB-438 (breast cancer), PC3 (prostate cancer), T47D (breast cancer), THP-1 (acute myeloid leukemia), U87 (glioblastoma), SHSY5Y Human neuroblastoma cells, cloned from a myeloma, Saos-2 cells (bone cancer), and the like.

In certain embodiments suitable cell lines include, but are not limited to, cell lines listed in Table 1.

TABLE 1
Illustrative, but non-limiting examples of cells that can be transfected
using the electroporation devices and methods described herein.

| Cell line | Organism | Origin tissue |
|---|---|---|
| 293-T | Human | Kidney (embryonic) |
| 3T3 cells | Mouse | Embryonic fibroblast |
| 4T1 | murine | breast |
| 721 | Human | Melanoma |
| 9L | Rat | Glioblastoma |
| A2780 | Human | Ovary |
| A2780ADR | Human | Ovary |
| A2780cis | Human | Ovary |
| A172 | Human | Glioblastoma |
| A20 | Murine | B lymphoma |
| A253 | Human | Head and neck carcinoma |
| A431 | Human | Skin epithelium |
| A-549 | Human | Lung carcinoma |
| ALC | Murine | Bone marrow |
| B16 | Murine | Melanoma |
| B35 | Rat | Neuroblastoma |
| BCP-1 cells | Human | PBMC |
| BEAS-2B | Human | Lung |
| bEnd.3 | Mouse | Brain/cerebral cortex |
| BHK-21 | Hamster | Kidney |
| BR 293 | Human | Breast |
| BxPC3 | Human | Pancreatic adenocarcinoma |
| C2C12 | Mouse | Myoblast cell line |
| C3H-10T1/2 | Mouse | Embryonic mesenchymal cell line |
| C6/36 | Asian tiger | Larval tissue |
| | mosquito | |
| C6 | Rat | Glioma |
| Cal-27 | Human | Tongue |
| CGR8 | Mouse | Embryonic Stem Cells |
| CHO | Hamster | Ovary |
| COR-L23 | Human | Lung |
| COR-L23/CPR | Human | Lung |
| COR-L23/5010 | Human | Lung |
| COR-L23/R23 | Human | Lung |
| COS-7 | Monkey | Kidney |
| COV-434 | Human | Ovary |
| CML T1 | Human | CML acute phase |
| CMT | Dog | Mammary gland |
| CT26 | Murine | Colorectal carcinoma |
| D17 | Canine | Osteosarcoma |
| DH82 | Canine | Histiocytosis |
| DU145 | Human | Androgen insensitive carcinoma |
| DuCaP | Human | Metastatic prostate cancer |
| E14Tg2a | Mouse | |
| EL4 | Mouse | |
| EM2 | Human | CML blast crisis |
| EM3 | Human | CML blast crisis |
| EMT6/AR1 | Mouse | Breast |
| EMT6/AR10.0 | Mouse | Breast |
| FM3 | Human | Metastatic lymph node |
| H1299 | Human | Lung |
| H69 | Human | Lung |
| HB54 | Hybridoma | Hybridoma |
| HB55 | Hybridoma | Hybridoma |
| HCA2 | Human | Fibroblast |
| HEK-293 | Human | Kidney (embryonic) |
| HeLa | Human | Cervical cancer |
| Hepa1c1c7 | Mouse | Hepatoma |
| High Five cells | Insect (moth) | Ovary |
| HL-60 | Human | Myeloblast |
| HMEC | Human | |
| HT-29 | Human | Colon epithelium |
| HUVEC | Human | Umbilical vein endothelium |
| Jurkat | Human | T cell leukemia |
| J558L cells | Mouse | Myeloma |
| JY cells | Human | Lymphoblastoid |
| K562 cells | Human | Lymphoblastoid |
| Ku812 | Human | Lymphoblastoid |
| KCL22 | Human | Lymphoblastoid |
| KG1 | Human | Lymphoblastoid |
| KYO1 | Human | Lymphoblastoid |
| LNCap | Human | Prostatic adenocarcinoma |
| Ma-Mel 1, 2, | Human | |
| 3 . . . 48 | | |
| MC-38 | Mouse | |
| MCF-7 | Human | Mammary gland |
| MCF-10A | Human | Mammary gland |
| MDA-MB-231 | Human | Breast |
| MDA-MB-468 | Human | Breast |
| MDA-MB-435 | Human | Breast |
| MDCK II | Dog | Kidney |
| MDCK II | Dog | Kidney |
| MG63 | Human | Bone |
| MOR/0.2R | Human | Lung |
| MONO-MAC 6 | Human | WBC |
| MRC5 | Human (foetal) | Lung |
| MTD-1A | Mouse | |
| MyEnd | Mouse | |
| NCI-H69/CPR | Human | Lung |
| NCI-H69/LX10 | Human | Lung |
| NCI-H69/LX20 | Human | Lung |
| NCI-H69/LX4 | Human | Lung |
| NIH-3T3 | Mouse | Embryo |
| NALM-1 | | Peripheral blood |
| NW-145 | | |
| OPCN/OPCT cell | | |
| lines | | |
| Peer | Human | T cell leukemia |
| PNT-1A/PNT 2 | | |
| Raji | human | B lymphoma |
| RBL cells | Rat | Leukemia |
| RenCa | Mouse | |
| RIN-5F | Mouse | Pancreas |
| RMA/RMAS | Mouse | |
| S2 | Insect | Late stage (20-24 hours old) |
| | | embryos |
| Saos-2 cells | Human | |
| Sf21 | Insect (moth) | Ovary |
| Sf9 | Insect (moth) | Ovary |
| SiHa | Human | Cervical cancer |
| SKBR3 | Human | |
| SKOV-3 | Human | |
| T2 | Human | |
| T-47D | Human | Mammary gland |
| T84 | Human | Colorectal carcinoma/Lung |
| | | metastasis |
| 293-T | Human | Kidney (embryonic) |
| 3T3 cells | Mouse | Embryonic fibroblast |
| 4T1 | murine | breast |
| 721 | Human | Melanoma |
| 9L | Rat | Glioblastoma |
| A2780 | Human | Ovary |
| A2780ADR | Human | Ovary |
| A2780cis | Human | Ovary |
| A172 | Human | Glioblastoma |
| A20 | Murine | B lymphoma |
| A253 | Human | Head and neck carcinoma |
| A431 | Human | Skin epithelium |
| A-549 | Human | Lung carcinoma |
| ALC | Murine | Bone marrow |
| B16 | Murine | Melanoma |
| B35 | Rat | Neuroblastoma |
| BCP-1 cells | Human | PBMC |
| BEAS-2B | Human | Lung |
| bEnd.3 | Mouse | Brain/cerebral cortex |
| BHK-21 | Hamster | Kidney |
| BR 293 | Human | Breast |
| BxPC3 | Human | Pancreatic adenocarcinoma |
| C2C12 | Mouse | Myoblast cell line |
| C3H-10T1/2 | Mouse | Embryonic mesenchymal cell line |
| C6/36 | Asian tiger | Larval tissue |
| | mosquito | |
| C6 | Rat | Glioma |
| Cal-27 | Human | Tongue |
| CHO | Hamster | Ovary |
| COR-L23 | Human | Lung |
| COR-L23/CPR | Human | Lung |
| COR-L23/5010 | Human | Lung |
| COR-L23/R23 | Human | Lung |
| COS-7 | Ape | Kidney |
| COV-434 | Human | Ovary |
| CML T1 | Human | CML acute phase |
| CMT | Dog | Mammary gland |
| CT26 | Murine | Colorectal carcinoma |
| D17 | Canine | Osteosarcoma |
| DH82 | Canine | Histiocytosis |
| DU145 | Human | Androgen insensitive carcinoma |
| DuCaP | Human | Metastatic prostate cancer |
| EL4 | Mouse | |
| EM2 | Human | CML blast crisis |
| EM3 | Human | CML blast crisis |
| EMT6/AR1 | Mouse | Breast |
| EMT6/AR10. 0 | Mouse | Breast |
| FM3 | Human | Metastatic lymph node |
| H1299 | Human | Lung |
| H69 | Human | Lung |
| HB54 | Hybridoma | Hybridoma |
| HB55 | Hybridoma | Hybridoma |
| HCA2 | Human | Fibroblast |
| HEK-293 | Human | Kidney (embryonic) |
| HeLa | Human | Cervical cancer |
| Hepa1c1c7 | Mouse | Hepatoma |
| High Five cells | Insect (moth) | Ovary |
| HL-60 | Human | Myeloblast |
| HMEC | Human | |
| HT-29 | Human | Colon epithelium |
| HUVEC | Human | Umbilical vein endothelium |
| Jurkat | Human | T cell leukemia |
| J558L cells | Mouse | Myeloma |
| JY cells | Human | Lymphoblastoid |
| K562 cells | Human | Lymphoblastoid |
| Ku812 | Human | Lymphoblastoid |
| KCL22 | Human | Lymphoblastoid |
| KG1 | Human | Lymphoblastoid |
| KYO1 | Human | Lymphoblastoid |
| LNCap | Human | Prostatic adenocarcinoma |
| Ma-Mel 1, 2, | Human | |
| 3 . . . 48 | | |
| MC-38 | Mouse | |
| MCF-7 | Human | Mammary gland |
| MCF-10A | Human | Mammary gland |
| MDA-MB-231 | Human | Breast |
| MDA-MB-468 | Human | Breast |
| MDA-MB-435 | Human | Breast |
| MDCK II | Dog | Kidney |
| MDCK II | Dog | Kidney |
| MG63 | Human | Bone |
| MOR/0.2R | Human | Lung |
| MONO-MAC 6 | Human | WBC |
| MRCS | Human (foetal) | Lung |
| MTD-1A | Mouse | |
| MyEnd | Mouse | |
| NCI-H69/CPR | Human | Lung |
| NCI-H69/LX10 | Human | Lung |
| NCI-H69/LX20 | Human | Lung |
| NCI-H69/LX4 | Human | Lung |
| NIH-3T3 | Mouse | Embryo |
| NALM-1 | | Peripheral blood |
| NW-145 | | |
| OPCN/OPCT cell | | |
| lines | | |
| Peer | Human | T cell leukemia |
| PNT-1A/PNT 2 | | |
| PTK2 | Rat Kangaroo | kidney |
| Raji | human | B lymphoma |
| RBL cells | Rat | Leukaemia |
| RenCa | Mouse | |
| RIN-5F | Mouse | Pancreas |
| RMA/RMAS | Mouse | |
| Saos-2 cells | Human | |
| Sf21 | Insect (moth) | Ovary |
| Sf9 | Insect (moth) | Ovary |
| SiHa | Human | Cervical cancer |
| SKBR3 | Human | |
| SKOV-3 | Human | |
| T2 | Human | |
| T-47D | Human | Mammary gland |
| T84 | Human | Colorectal carcinoma/Lung |
| | | metastasis |
| THP1 cell line | Human | Monocyte |
| U373 | Human | Glioblastoma-astrocytoma |
| U87 | Human | Glioblastoma-astrocytoma |
| U937 | Human | Leukemic monocytic lymphoma |
| VCaP | Human | Metastatic prostate cancer |
| Vero cells | African green | Kidney epithelium |
| | monkey | |
| WM39 | Human | Skin |
| WT-49 | Human | Lymphoblastoid |
| X63 | Mouse | Melanoma |
| YAC-1 | Mouse | Lymphoma |
| YAR | Human | B cell |

It will be appreciated that the foregoing cell types are intended to be illustrative and non-limiting. It will be recognized that numerous other eukaryotic cell types can readily be used with the electroporation devices and methods described herein.

### REFERENCES

1. Lingqian Chang, Paul Bertani, Daniel Gallego-Perez, Zhaogang Yang, Feng Chen, Chiling Chiang, Veysi Malkoc, Tairong Kuang, Keliang Gao, L. James Lee and Wu Lu “3D nanochannel electroporation for high-throughput cell transfection with high uniformity and dosage control” Nanoscale, 8, 243-252 (2016).

2. Hang Lu, Martin A Schmidt, Klays F. Jensen “A microfluidic electroporation device for cell lysis” Lab Chip, 5, 23-29 (2005).

3. Stefano Vassanelli and Giorgio Cellere “Biochip electroporator and its use in multi-site, single-cell electroporation” US Patent number: U.S. Pat. No.: 8,017,367.

4. Armon Sharei, Janet Zoldan, Andrea Adamo, Woo Young Sim, Nahyun Cho, Emily Jackson, Shirley Mao, Sabine Schneider, Min-Joon Han, Abigail Lytton-Jean, Pamela A. Basto, Siddharth Jhunjhunwala, Jungmin Lee, Daniel A. Heller, Jeon Woong Kang, George C. Hartoularos, Kwang-Soo Kim, Daniel G. Anderson, Robert Langer, and Klays F. Jensena, “A vector-free microfluidic platform for intracellular delivery,” Proc Natl Acad Sci, 110, 2081-2087, 2013.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A device for parallel single cell electroporation, said device comprising:
a substrate comprising a plurality of through holes forming substantially parallel channels and a plurality of electrodes disposed so that each electrode comprising said plurality of electrodes intersects a subset of said plurality of holes and is configured to apply a voltage to or across the edges of said holes.

2. The device of claim 1, wherein said plurality of through holes comprises through holes disposed in a regular array and said plurality of electrodes comprises rows of electrodes disposed between rows of said holes each electrode intersecting a plurality of holes that comprises a row of holes.

3. The device of claim 1, wherein electrodes comprising said plurality of electrodes are covered with a dielectric material.

4. The device of claim 3, wherein said dielectric material is selected from the group consisting of an oxide, a photoresist, and polyimide.

5. (canceled)

6. The device of claim 1, wherein:
said plurality of holes form parallel channels having an average or median length ranging from about 1 μm up to about100 μm, or from about 5 μm up to about 50 μm, or from about 10 μm up to about 40 μm; and/or the average or median diameter of said plurality of holes ranges from about 5 μm up to about 50 μm, or from about 10 μm up to about 40 μm, or from about 15 μm up to about 30 μm, or up to about 20 μm, and/or
said device comprises at least 500 through holes, or at least 1000 through holes, or at least 2000 through holes, or at least 3000 through holes, or at least 4,000 through holes, or at least 5,000 through holes, or at least 6000 through holes, or at least 7, 000 through holes, or at least 8,000 through holes, or at least 9,000 through holes, or at least 10,000 through holes, or at least 15,000 through holes, or at least 20,000 through holes, or at least 50,000 through holes, or at least 100,000 through holes, or at least 250,000 through holes, or at least 500,000 through holes, or at least 750,000 through holes, or at least 1,000,000 through holes; and/or
said through holes are disposed in an area ranging from about 0.5 cm², or from about 1 cm², up to about 10 cm², or up to about 8 cm², or up to about 6 cm², or up to about 5 cm², or up to about 4 cm², or up to about 3cm², or up to about 2 cm², or up to about 1.5 cm²; and/or
said device comprises at least about 500 holes/cm², or at least about 1000 holes/cm², or at least about 2000 holes/cm², or at least about 3000 holes/cm², or at least about 4,000 holes, or at least about 5,000 holes/cm², or at least about 6000 holes/cm², or at least about 7, or 000 holes/cm², or at least about 8,000 holes/cm², or at least about 9,000 holes/cm², or at least about 10,000 holes/cm², or at least about 15,000 holes/cm², or at least about 20,000 holes/cm², or at least about 25,000 holes/cm², or at least about 30,000 holes/cm², or at least about 35,000 holes/cm², or at least about 40,000 holes/cm².

7. (canceled)

8. The device of claim 1, wherein said through holes are configured to contain no more than 15 cells, or no more than 10 cells, or no more than 5 cells, or no more than 4 cells, or no more than 3 cells, or no more than 2 cells, or only one cell at a time.

9-11. (canceled)

12. The device of claim 1, wherein said substrate comprises a silicon substrate.

13. The device of claim 1, wherein said electrodes comprise a metal or metal alloy.

14. The device of claim 1, wherein said electrodes comprise a material selected from the group consisting of gold, silver, copper, graphite, titanium, brass, platinum, graphene, indium tin oxide (ITO), and carbon nanotube(s).

15. The device of claim 1, wherein:
the width of said electrode ranges from about 5 μm, or from about 10 μm, or from about 15 μm, or from about 20 μm up to about 500 μm, or from about 20 μm, or from about 30 μm, or from about 40 μm, or from about 50 μm up to about 500 μm, or up to about 400 μm, or up to about 300 μm, or up to about 200 μm, or up to about 150 μm; and/or
the thickness of said electrode ranges from about 0.01 μm, or from about 0.05 μm, or from about 0.1 μm, or from about 0.2 μm, or from about 0.5 μm, or from about 1 μm, or from about 2 μm, or from about 3 μm, or from aobut 4 μm, or from about 5 μm, or from about 10 μm up to about 100 μm, or up to about 50 μm, or up to about 40 μm, or up to about 30 μm, or up to about 20 μm.

16. (canceled)

17. The device of claim 1, wherein said device further comprises a supporting structure comprising passages configured to permit fluid passage through said supporting structure and into said plurality of holes.

18. The device of claim 17, wherein said supporting structure comprises a honeycomb structure disposed on said substrate so that cells to be transfected pass through said honeycomb structure before entering holes comprising said plurality of through holes.

19-20. (canceled)

21. The device of claim 18, wherein:
said electrodes are disposed as a first layer on the substrate comprising said plurality of holes;
a dielectric layer is disposed on the top of said electrodes; and
said honeycomb is comprises a second layer disposed on the opposite side of said substrate that the side on which said electrodes are disposed.

22. The device of claim 1, wherein said electrodes are operably coupled to a power supply.

23. The device of claim 22, wherein:
said power supply provides a voltage ranging from about 1V, or from about 2V, or from about 3V, or from about 4V, or from about 5V up to about 50V, or up to about 40V, or up to about 30V, or up to about 20V, or up to about 15V; and/or
said power supply is configured to provide an AC voltage; and/or
said AC voltage ranges in frequency from about 10 Hz, or from about 100 Hz, or from about 1 kHz, or from about 10 kHz, up to about 1 MHz, or up to about 5 MHz, or up to about 10 MHz, or up to about 50 MHz.

24-28. (canceled)

29. The device of claim 1, wherein:
said device is in fluid communication with a chamber containing cells to be electroporated; and/or
said device is in fluid communication with a chamber containing a reagent (cargo) that is to be electroporated into said cells.

30-32. (canceled)

33. The device of claim 29, wherein said chamber(s) are pressurized to force fluid containing said cells through said plurality of holes.

34. The device of claim 29, wherein said chamber(s) are chambers of a syringe or syringe pump.

35. A method of making an electroporation device of claim 1, said method comprising:
providing a substrate;
backside etching of said substrate to form a honeycomb structure;
patterning and deposition of said plurality of electrodes on the front side surface of said substrate;
etching through holes through said substrate and into the honeycomb structure.

36-41. (canceled)

42. The method of claim 35, wherein said method comprising depositing a dielectric layer on top of said electrodes.

43. A method of delivering a cargo into a plurality of cells, said method comprising:
providing cells in solution containing the cargo that is to be electroporated into said cells; and
passing said cells through the plurality of through holes in a device of claim 1, while applying a voltage to said electrodes whereby said cargo is electroporated into said cells.

44-49. (canceled)

50. The method of claim 43, wherein:
said voltage ranges from about 1V, or from about 2V, or from about 3V, or from about 4V, or from about 5V up to about 50V, or up to about 40V, or up to about 30V, or up to about 20V, or up to about 15V; and/or
said voltage is an applied AC voltage; and/or
said voltage ranges in frequency from about 10 Hz, or from about 100 Hz, or from about 1 kHz, or from about 10 kHz, up to about 1 MHz, or up to about 5 MHz, or up to about 10 MHz, or up to about 50 mHz.

51-55. (canceled)

56. The method of claim 43, wherein said cargo comprises one or moieties selected from the group consisting of a dye, a nucleic acid (e.g., RNA, DNA), a protein (including, but not limited to, antibodies, intrabodies, enzymes (e.g., kinases, proteases, helicases, phosphorylates, etc.), signaling molecules, and the like), a vector (e.g., a plasmid, a phagemid, bacteriophage vector, cosmid, etc.), a natural chromosome or chromosome fragment, a synthetic chromosome or chromosome fragment, a virus particle, a bacterium, an intracellular fungus, an intracellular protozoan, an organelle, various particles (e.g., nanoparticles, polymeric particles, drug-carrying particles, quantum dots, etc.), small organic molecules, probes, and labels.

57-66. (canceled)

67. The method of claim 43, wherein said cells comprise a plant cell, a yeast cell, an algal cell, a fungal cell, an invertebrate animal cell (e.g., an insect cell), and a vertebrate animal cell.

68-76. (canceled)

77. The method of claim 43, wherein:
said device is operated at a flow rate that ranges from about 0.1 mL/min, or from about 0.5 mL/min, or from about 1.0 mL/min up to about 20 mL/min, or up to about 15 mL/min, or up to about 10 mL/min, or up to about 5 mL/min, or up to about 4 mL/min, or up to about 3 mL/min, or up to about 2 mL/min, or up to about 1.5 mL/min, or at about 1.12 mL/min; and/or
said cells are provided in said device at a density ranging from about 10⁵ cells/mL up to about 10⁹ cells/mL, or from about 10⁶ cells/mL up to about 10⁸ cells/mL, or about 10⁷ cells/mL, and/or
said device transfects cells at a delivery efficiency of at least about 10%, or at least about 20%, or at least 30%, or at least about 40%, or at least about 50%, or at least about 60%, at least about 70%, or at least about 80%, or at least about 90%; and/or
said device transfects cells with a cell viability of at least about 40%, or at least about 50%, or at least about 60%, at least about 70%, or at least about 80%, or at least about 90%; and/or
said method delivers cargos in up to 10 million cells/min on a 1 cm² chip.

78-81. (canceled)
